(19) United States
(12) Plant Patent Application Publication
(10) Pub. No.: US 2003/0005500 P1
(43) Pub. Date: Jan. 2, 2003

(54) Avocado Plant named 'Llanos Hass'

(76) Inventors: Anthony Phillip Llanos, Kwinana (AU); Cassandra Ann Llanos, Kwinana (AU)

Correspondence Address: CHRISTIE, PARKER & HALE, LLP, 350 WEST COLORADO BOULEVARD, SUITE 500, PASADENA, CA, 91105, US

(21) Appl. No.: 09/876,599

(22) Filed: Jun. 7, 2001

(51) Int. Cl. A01H 5/00

(52) U.S. Cl. PLT/200

(57) ABSTRACT

Described is a new Avocado variety whose fruit matures at a minimum of approximately 4 to 6 weeks earlier than the variety ‘Hass’.

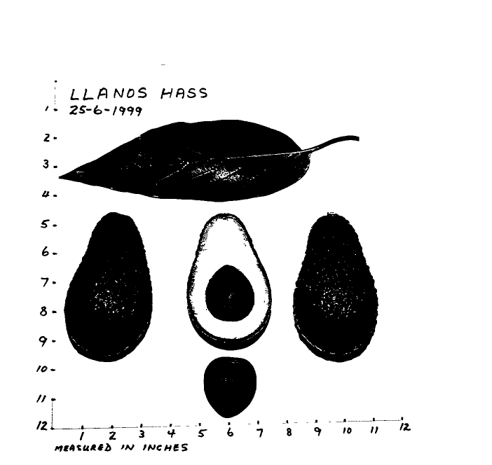

### BACKGROUND OF THE INVENTION

Avocado plants in cultivation are mainly varieties of Persea Americano. The new variety was developed by open pollination followed by seedling selection: an open pollinated seedling was selected at applicant's property in Kwinana, Hope Valley, Western Australia. which displayed precocious and consistent fruit, and which was an improvement to the variety ‘Hass’ inasmuch as it consistently matured earlier than ‘Hass’. DNA profiling has shown that ‘Hass’ is likely to be one of the parents of the new variety. Propagation by vegetative grafting onto rootstocks in Kwinana, Hope Valley, Western Australia has demonstrated that the characteristics of the new variety are transmitted through succeeding generations of asexual reproduction.

### SUMMARY OF THE INVENTION

The present invention is a new and distinctive Avocado plant having precocious and consistent fruit set similar to that of its parent variety, ‘Hass’, but matures consistently approximately 4 to 6 weeks earlier than that of its parent variety ‘Hass.’

The new variety was asexually reproduced by vegetative grafting onto Guatemalan Avocado seedling rootstocks and then advantageously planted in soil consisting of deep sands.

### BRIEF DESCRIPTION OF ILLUSTRATION(S)

The accompanying photo illustration shows a typical fruit and leaf of the new variety depicted in color as true as possible in a color illustration of this character.

### DESCRIPTION OF THE NEW VARIETY

The new variety ‘LLANOS HASS’ is vigorous, upright with a medium to large height. Anthocyanin is not present in the stems of young shoots. The leaflets are large, horizontal, long and narrow with blades folding concave with a lanceolate shape. The tips of the leaflets are acute and anthocyanin is present in newly emerged leaves. The following description is of plants grown in Kwinana, Hope Valley, Western Australia with color descriptions in accordance with the Color Chart of The Royal Horticultural Society, London (3^{rd} ed. 1995). The plants described were grafted in 1996 onto rootstock as previously described.

All dimensions are in millimeters, weights in grams (unless otherwise stated).

COMPARATIVE TRIAL

| | |
|---|---|
| COMPARISON VARIETY: | |
| ‘HASS’ | |
| Location: | Kwinana, Hope Valley, Western Australia |
| | (Zone 50) approximately 30 Km south of |
| | Perth. |
| Condition: | Scion wood from the original “Llanos |
| | Hass’ seedling tree was grafted onto |
| | Guatemalan seedling rootstocks, trees |
| | were planted in 1996 at spacings of |
| | 5.0 m × 2.5 m to a total of 100 trees. |
| | ‘Hass’ trees grafted onto Guatemalan |
| | seedling rootstocks were planted randomly |
| | within the “Llanos Hass’ block in 1997 to |
| | a total of 10 trees. The plants were grown |
| | in the field, soil being deep sands; all |
| | trees were managed in the same manner |
| | with regard to irrigation and nutrition. |
| | Irrigation using mini sprinklers aimed to |
| | wet 100% of soil in the drip zone. |
| | Nutrition requirements were based on |
| | annual leaf analysis and applied by |
| | fertigation. |
| Trial design: | The trial set up was of a completely |
| | randomized design. |
| Measurements: | Samples were randomly collected from |
| | trees selected at random for analysis. |

CHARACTERISTICS

| | |
|---|---|
| Plant: | Habit vigorous, upright, height medium to |
| | large. |
| Stem: | Anthocyanin not present in young shoots. |
| Leaf: | Large size (183 mm × 71 mm), shape |
| | lanceolate (length/width ratio about 2.6) |
| Attitude: | Horizontal, blade folding concave |
| Length: | Long |
| Width: | Narrow |
| Tip: | Acute, anthocyanin present in newly |
| | emerged leaves, anise aroma not present |
| | when crushed. |
| Inflorescence: | Size axis length medium, Type ‘B’. |
| Flower: | Habit late, pubescence on sepals medium. |
| Mature fruit: | Early maturing, size small (97 mm × |
| | 64 mm), shape base rounded stylar region |
| | slightly depressed, length: diameter ratio |
| | medium (1.5:1), stalk cavity present, relief |
| | of surface rough. |
| Pedicel: | Length medium, shape cylindrical, |
| | nailhead shape present, pedicel/peduncle |
| | combined length medium (81 mm). |
| Ripe fruit: | Color of skin purple black, thickness of |
| | skin thick (1.75 mm), texture of skin |
| | leathery, adherence of skin to flesh |
| | medium, main flesh color yellow, wide |
| | green layer of flesh next to skin, fibres |
| | in flesh inconspicuous, flesh texture |
| | smooth, seed set into cavity tight. |
| | Seed: size compared to fruit large (flesh |
| | weight:seed weight ratio 3.7:1), shape in |
| | longitudinal section base flattened, apex |
| | conical. |

COMPARISON OF PERSEA VARIETIES

| | ‘Llanos Hass’ | *‘Hass’ |
|---|---|---|
| YOUNG SHOOT | | |
| Color | green | green |
| Anthocyanin coloration | absent | absent |
| Color of lenticels | red | green |
| YOUNG LEAF: | | |
| Anthocyanin coloration | present | present |
| Color of petiole pubescence | white | white |
| LEAF: | | |
| Attitude | horizontal | horizontal |
| Blade/folding | concave | concave |
| Blade size | large | large |
| Blade Length (mm) | | |
| Mean | 183 | 165 |
| Std. Deviation | 21 | 25 |
| LSD/sig | 9 | pS0.01 |
| Blade Width (mm) | | |
| Mean | 71 | 76 |
| Std. Deviation | 10 | 9 |
| LSD/sig | 4 | pS0.01 |
| Blade Length/Width Ratio | | |
| Mean | 2.6 | 2.2 |
| Std. Deviation | 0.3 | 0.4 |
| LSD/sig | 0.1 | PS0.01 |
| Blade shape | lanceolate | elliptical |
| Blade—shape of tip | acute | acute |
| Blade undulation of margin | weak | weak |
| Blade anise aroma | absent | absent |
| Inflorescence: | | |
| Length of Axis | medium | long |
| Color of Lenticels | green | green |
| Flowering type | Type B | Type A |
| Flower: | | |
| Pubescence of sepal | present | present |
| Density Pubescence of sepal | medium | medium |
| Mature fruit: | | |
| Size | small | small |
| Length (mm) | | |
| Mean | 97 | 100 |
| Std. Deviation | 10 | 11 |
| LSD/sig | 7.0 | ns |
| Diameter (widest point −mm) | | |
| Mean | 64 | 66 |
| Std. Deviation | 6 | 7 |
| LSD/sig | 4.0 | ns |
| Length/Diameter ratio | | |
| Mean | 1.5 | 1.5 |
| Std. Deviation | 0.1 | 0.1 |
| LSD/sig | 0.1 | ns |
| Shape of basal part of fruit | rounded | rounded |
| Stalk cavity | present | present |
| shape of stylar region | slightly | slightly |
| | depressed | depressed |
| Relief of surface | rough | rough |
| Pedicel: | | |
| Length | medium | medium |
| Shape | cylindrical | cylindrical |
| “Nailhead” Shape | present | present |
| color | green | yellow/green |
| Pedicel/Pedunle combined Length | | |
| Mean | 81 | 123 |
| Std. Deviation | 15 | 33 |
| LSD/sig | 13.8 | PS0.01 |
| Ripe fruit: | | |
| Color of skin | purple black | purple black |
| Thickness of skin (mm) | thick | thick |
| Mean | 1.75 | 1.42 |
| Std. Deviation | 0.16 | 0.22 |
| LSD/sig | 0.20 | pS0.01 |
| Texture of skin | leathery | corky |
| Adherence of skin to flesh | medium | medium |
| Main color of flesh | yellow | yellow |
| Color of flesh next to skin | green | green |
| Width of colored layer of flesh | wide | medium |
| next to skin | | |
| Texture of flesh | smooth | smooth |
| Firmness of flesh | medium | medium |
| Anise aroma of flesh | absent | absent |
| Setting of seed in cavity | tight | tight |
| Size compared to fruit size | large | medium |
| Flesh: seed to weight ratio | | |
| Mean | 3.7 | 4.8 |
| Std. Deviation | 0.6 | 1.0 |
| LSD/sig | 0.5 | PS0.01 |
| Seed: shape in longitudal section | base flattened, | ovate |
| | apex conical | |
| Time of flowering: | late | late |
| Time of fruit maturity for harvesting: | early | late |

We claim:

1. A new Avocado plant of the variety substantially as shown and described.
